(19) United States
(12) Patent Application Publication
(10) Pub. No.: US 2017/0368015 A1
(43) Pub. Date: Dec. 28, 2017

(54) NOVEL COMPOUND AND ANTIVIRAL AGENT CONTAINING SAME AS ACTIVE INGREDIENT

(71) Applicant: NATIONAL UNIVERSITY CORPORATION CHIBA UNIVERSITY, Chiba-shi (JP)

(72) Inventors: Hiroshi SHIRASAWA, Chiba-shi (JP); Takayoshi ARAI, Chiba-shi (JP); Yutaka TAMURA, Chiba-shi (JP); Kengo SAITO, Chiba-shi (JP); Akiko SUGANAMI, Chiba-shi (JP); Yoshifumi OHNO, Chiba-shi (JP); Akira YANAGISAWA, Chiba-shi (JP); Shoji MATSUMOTO, Chiba-shi (JP); Tetsuhiro NEMOTO, Chiba-shi (JP); Ouji WATANABE, Chiba-shi (JP)

(73) Assignee: NATIONAL UNIVERSITY CORPORATION CHIBA UNIVERSITY, Chiba-shi (JP)

(21) Appl. No.: 15/539,846

(22) PCT Filed: Dec. 25, 2015
(86) PCT No.: PCT/JP2015/086240

§ 371 Date: Jun. 26, 2017

(30) Foreign Application Priority Data
Dec. 26, 2014 (JP) 2014-264513

(51) Int. Cl. A61K 31/277; A61K 31/555; A61K 31/4439; A61K 31/4245; A61K 31/407; A61K 31/136; A61K 31/404; A61K 31/4035; A61K 31/401; A61K 31/198; A61K 31/675; A61K 31/4162

(52) U.S. Cl. CPC A61K 31/277; A61K 31/675; A61K 31/555; A61K 31/4439; A61K 31/4245; A61K 31/4162; A61K 31/136; A61K 31/404; A61K 31/4035; A61K 31/401; A61K 31/198; A61K 31/407

(57) ABSTRACT

An object of the invention is to provide a compound that can be utilized as an antiviral agent, in particular as an anti-RNA viral agent, and especially as an anti-RS viral agent. The invention provides a compound indicated by Formula (1), wherein R1 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl, or optionally substituted aryl; R2 each independently represent hydrogen, C1-C6 alkyl, C1-C6 halogenoalkyl, C2-C5 alkenyl, C3-C6 cycloalkyl, optionally substituted aryl or heterocyclic group; and one or more R1 may be present in the same ring, an isomer thereof, a pharmaceutically acceptable salt thereof, or a mixture of these. The compounds provided by the invention are useful as drugs for the prevention or treatment of infectious diseases by virus, especially RS virus, and in particular infectious diseases in the lower airways (e.g., bronchiolitis, pneumonia, etc.).

### CROSS-REFERENCE TO RELATED APPLICATIONS

This application is a U.S. National Phase Application of PCT International Application PCT/JP2015/086240, filed Dec. 25, 2015 which claims priority to Japanese Application No. 2014-264513, filed Dec. 26, 2014, the contents of which are incorporated herein by reference in their entireties for all purposes.

### TECHNICAL FIELD

The present invention relates to a compound having antiviral activity, particularly anti-RS viral activity, and to an antiviral agent containing such compound as an active ingredient.

### BACKGROUND OF THE INVENTION

As an RNA virus whose virion has a negative-sense single-stranded RNA as a genome, Orthomyxoviridae viruses including influenza virus, and Paramyxoviridae viruses including mumps virus that causes so-called epidemic parotiditis are known. These viruses have a structure that an RNA genome is wrapped with an envelope.

It is well known that influenza virus is a pathogen that causes so-called “influenza flu”. Examples of viruses included in Paramyxoviridae include canine distemper virus, rinderpest virus, measles virus, newcastle disease virus, RS virus (human respiratory syncytial virus) in addition to the aforementioned mumps virus, all of which are pathogens on human or mammals.

Among the viruses recited above, RS virus (respiratory syncytial virus) causes respiratory infectious diseases accompanied by rhinitis or cough, and is positioned as the most important respiratory pathogen in infancy and early childhood. In particular, for infants suffering from innate cardiac defects, bronchopulmonary hypoplasia, immunodeficient diseases and the like, RS virus causes infectious diseases that are critical and highly possibly lethal. Also in an adult who is in an immunosuppressive state after bone marrow transplantation or organ transplantation, RS virus can be a dangerous pathogen.

Conventional treatments for RS viral infectious diseases are actually limited to symptomatic treatments such as rehydration, oxygen supplementation, and assisted respiration. While ribanvirin which is a nucleic acid derivative has been used as a therapeutic drug for RS viral infectious diseases, there is a demand for a therapeutic drug for viral infectious diseases that is more effective and safer.

As a therapeutic drug for RS viral infectious diseases, various substances including low molecular organic compounds (for example, Patent Document 1, Patent Document 2, and Patent Document 3), vegetable-derived extracts (for example, Patent Document 4, and Patent Document 5), vaccines (for example, Patent Document 6), and monoclonal antibodies (for example, Patent Document 7) have been proposed. However, there is still a need for the drugs that are effective as an anti-RS viral agent.

### CITATION LIST

### Patent Literatures

Patent Literature 1: JP2003-512384 W

Patent Literature 2: Re-publication of PCT International Publication WO01/060834 A

Patent Literature 3: JP2005-089450 A

Patent Literature 4: Re-publication of PCT International Publication WO2010/005010 A

Patent Literature 5: JP2004-083487 A

Patent Literature 6: JP2001-517448 W

Patent Literature 7: JP2002-543822 W

### SUMMARY OF THE INVENTION

### Technical Problem

It is an object of the present invention to provide a compound that can be utilized as an antiviral agent, in particularly as an anti-RNA viral agent, and especially as an anti-RS viral agent.

### Solution to Problem

The present inventors found several compounds that can be utilized as an antiviral agent, in particular as an anti-RNA viral agent, and especially as an anti-RS viral agent from a compound library containing novel compounds and known compounds, and accomplished the present invention shown below.

(1). A compound indicated by the following Formula (1):

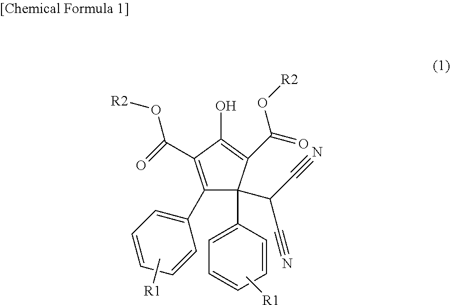

wherein R1 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R2 each independently represent hydrogen, C1-C6 alkyl, C1-C6 halogenoalkyl, C2-C5 alkenyl, C3-C6 cycloalkyl, optionally substituted aryl or heterocyclic group, and one or more R1 may be present in the same ring,
an isomer thereof, a pharmaceutically acceptable salt thereof, or a mixture of these.
(2). A compound indicated by the following Formula (2):

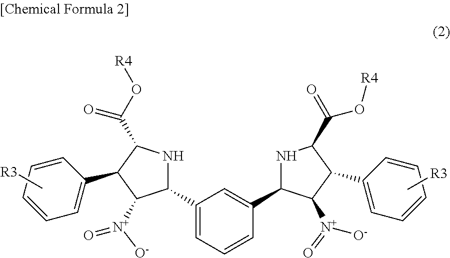

wherein R3 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R4 each independently represent hydrogen, C1-C6 alkyl, C1-C6 halogenoalkyl, C2-C5 alkenyl, C3-C6 cycloalkyl, or optionally substituted aryl, and one or more R3 may be present in the same ring,
an isomer thereof, a pharmaceutically acceptable salt thereof, or a mixture of these.
(3). A drug comprising as an active ingredient a compound selected from the group consisting of the following compounds:

the compound indicated by Formula (1) according to above (1);

the compound indicated by Formula (2) according to above (2);

Compound 3
2-(5-(5-(5-ferrocenylthiophene-2-yl)-1-phenyl-1H-pyrrol-2-yl)thiophe ne-2-yl)ethene-1,1,2-tricarbonitrile;

Compound 4
(R)-2-amino-2-(naphthalene-1-yl) acetic acid;

Compound 5
N1,N2-diphenylethane-1,2-diamine;

Compound 6
N1,N2-bis(4-methoxyphenyl)ethane-1,2-diamine;

Compound 7
N1,N2-bis(4-chlorophenyl)ethane-1,2-diamine;

Compound 8
(4S,11R)-1-phenyl-4-((phenylamino)methyl)-1,2,3,4,6-pentahydrobenzo[3,4][1,2]azaphosphoro [1,2-a][1,3,2]diazaphosphinine 11-oxide;

Compound 9
(S)-3-(1H-indole-3-yl)-1-methyl-3-(nitromethyl)indoline-2-one;

Compound 10
(S)-1,5-dimethyl-3-(7-methyl-1H-indole-3-yl)-3-(nitromethyl)indoline-2-one;

Compound 11
2,6-bis((2R,4S,5S)-1-benzyl-4,5-diphenylimidazolidine-2-yl)pyridine;

Compound 12
tert-butyl(2′R,3R,4′S,5′S)-4′-(4-bromophenyl)-2-oxo-2′-phenylspiro[i ndoline-3,3′-pyrrolidine]-5′-carboxylate;

Compound 13
tert-butyl(2′R,3R,4′5,5′S)-2-oxo-2′-phenyl-4′-(p-tolyl)spiro[indolin e-3,3′-pyrrolidine]-5′-carboxylate;

Compound 14
(E)-1-allyl-3-(nitromethylene)indoline-2-one;

Compound 15
2,4-dibromo-6-((E)-(((1R,2R)-2-(isoindoline-2-yl)-1,2-diphenylethyl) imino)methyl)phenol;

Compound 16
2-(phenyl((4-(trifluoromethoxy)phenyl)amino)methyl)cycloheptane-1-on e;

Compound 17
ethyl(R)-1-(3-chlorophenyl)-5-oxo-6,7-dihydro-1H,5H-pyrazolo[1,2-a]p yrazole-2-carboxylate;

Compound 18
ethyl(R)-1-(4-methoxyphenyl)-5-oxo-6,7-dihydro-1H,5H-pyrazolo[1,2-a] pyrazole-2-carboxylate;

Compound 19
ethyl(R)-1-cyclohexyl-5-oxo-6,7-dihydro-1H,5H-pyrazolo[1,2-a]pyrazol e-2-carboxylate;

Compound 20
methyl(2S,3R,4S,5S)-4-nitro-3,5-diphenylpyrrolidine-2-carboxylate;

Compound 21
2,5-diphenyl-1,3,4-oxadiazole; and

Compound 22
ethyl7,7-dimethyl-5-oxo-1-phenyl-6,7-dihydro-1H,5H-pyrazolo[1,2-a]pyrazole-2-carboxyl ate, an isomer thereof, a pharmaceutically acceptable salt thereof, or a mixture of these.
   (4). The drug according to above (3), wherein the drug is an anti-RNA viral agent.
   (5). The drug according to above (4), wherein the drug is an anti-RS viral agent.

### Advantageous Effects of Invention

The present invention provides a prophylactic or therapeutic agent for infectious diseases by virus, especially RS virus, and in particular infectious diseases in lower airways (e.g., bronchiolitis, pneumonia, etc.), or a prophylactic or therapeutic method for infectious diseases by RS virus, and in particular infectious diseases in lower airways (e.g., bronchiolitis, pneumonia, etc.) comprising administering a human or an animal with the prophylactic or therapeutic agent.

### DETAILED DESCRIPTION OF THE INVENTION

### Definitions

The term “halogen” herein means any atom selected from the group consisting of chlorine, fluorine, bromine and iodine.

The term “C1-C6 alkyl” means a straight-chain or branched-chain saturated hydrocarbon group having 1 to 6 carbon atoms, and examples thereof include, but are not limited to, methyl, ethyl, n-propyl, isopropyl, n-butyl, isobutyl, sec-butyl, tert-butyl, n-pentyl, isopentyl, sec-pentyl, tert-isopentyl, n-hexyl, isohexyl, sec-hexyl, and tert-isohexyl.

The term “C1-C6 alkoxyl” means an alkyl group having 1 to 6 carbon atoms that are bound to the molecule via an oxygen atom, and examples thereof include, but are not limited to, methoxy, ethoxy, n-propoxy, isopropoxy, n-butoxy, isobutoxy, sec-butoxy, tert-butoxy, n-pentyloxy, isopentyloxy, sec-pentyloxy, tert-pentyloxy, n-hexyloxy, isohexyloxy, sec-hexyloxy, and tert-hexyloxy.

The term “C1-C6 halogenoalkyl” means alkyl having 1 to 6 carbon atoms substituted by a halogen, and examples thereof include, but are not limited to, fluoromethyl, chloromethyl, bromomethyl, iodemethyl, dichloromethyl, trifluoromethyl and trichloromethyl, chloroethyl, dichloroethyl, trichloroethyl and chloropropyl and the like, mono, di or tri-substituted methyl halide.

The term “C1-C6 alkoxycarbonyl” represents a group having a structure: —COOR (R represents a C1-C6 alkyl), and examples thereof include, but are not limited to, methoxycarbonyl, ethoxycarbonyl, n-propoxycarbonyl, isopropoxycarbonyl, n-butoxycarbonyl, isobutoxycarbonyl, sec-butoxycarbonyl, tert-butoxycarbonyl, n-pentyloxycarbonyl, isopentyloxycarbonyl, sec-pentyloxycarbonyl, tert-pentyloxycarbonyl, n-hexyloxycarbonyl, isohexyloxycarbonyl, sec-hexyloxycarbonyl, and tert-hexyloxycarbonyl.

The term “C1-C6 alkylamino” means a group having a structure: —NH—(C1-C6 alkyl), or N—(C1-C6 alkyl)₂ᵣ and examples thereof include, but are not limited to, methylamino, ethylamino, propylamino, butylamino, pentylamino, hexylamino, dimethylamino, diethylamino and methylethylamino.

The term “C2-C5 alkenyl” means a hydrocarbon group having 2 to 5 carbon atoms and one unsaturated double bond, and examples thereof include, but are not limited to, vinyl, and allyl.

The term “C3-C6 cycloalkyl” means a group selected from the group consisting of cyclopropyl, cyclobutyl, cyclopentyl and cyclohexyl.

The term “aryl” means carbocyclic aromatic groups and heterocyclic aromatic groups, and examples thereof include, but are not limited to, phenyl, 1 or 2-naphthyl, fluorenyl, (1,2)-dihydronaphthyl, indenyl, indanyl, thienyl, benzothienyl, and thienopyridyl. The term “optionally substituted aryl” means carbocyclic aromatic groups and heterocyclic aromatic groups that may be substituted by halogen, C1-C6 alkyl, C1-C6 alkoxy, C1-C6 halogenoalkyl, C1-C6 alkoxycarbonyl, C1-C6 alkylamino and/or C2-C5 alkenyl.

The term “isomer” means enantiomers, diastereomers (including cis-trans isomers), conformational isomers, cis-trans isomers in double bond that can exist in the compound for which the term is used.

### Compound of Formula (1)

The first aspect of the present invention relates to a compound indicated by the following Formula (1), an isomer thereof, a pharmaceutically acceptable salt thereof, or a mixture of these.

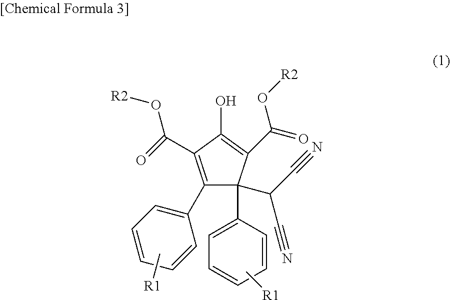

In Formula (1), R1 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R2 each independently represent hydrogen, C1-C6 alkyl, C1-C6 halogenoalkyl, C2-C5 alkenyl, C3-C6 cycloalkyl, optionally substituted aryl or heterocyclic group. One or more R1 may be present in the same ring.

A preferred substituent in the compound indicated by Formula (1) is hydrogen for each R1, and is methyl for each R2. A preferred compound of Formula (1) counting conformation is dimethyl 5-(dicyanomethyl)-2-hydroxy-4,5-diphenylcyclopenta-1,3-diene-1,3-dic arboxylate (hereinafter, indicated by Compound 1 (No. 150)).

The compound indicated by Formula (1) can be produced, for the case of Compound 1 (No. 150), by reacting dimethyl 2-oxo-4,5-diphenylcyclopenta-3,5-diene-1,3-dicarboxylate, malononitrile and aluminum oxide in an appropriate solvent such as chloroform.

The solvent used in the aforementioned production method is not particularly limited unless it adversely affects on the reaction, and examples of the solvent besides chloroform include aromatic hydrocarbons such as methylene chloride, benzene, toluene and xylene, ethers such as dioxane, tetrahydrofuran, anisole, diethyleneglycol diethylether, and dimethyl cellosolve, nitriles such as acetonitrile, amides such as N,N-dimethylacetamide, alcohols such as methanol, ethanol, and propanol, sulfoxides such as dimethyl sulfoxide, and water. These solvents can be used singly or in combination of two or more kinds.

In the production method of a compound indicated by Formula (1), each compound may be protected with an appropriate protective agent that is known by a person skilled in the art as is necessary. Such a protective agent and deprotecting reaction can be appropriately selected and used by a person skilled in the art according to the substituent to be protected.

### Compound of Formula (2)

The second aspect of the present invention relates to a compound indicated by the following Formula (2), an isomer thereof, a pharmaceutically acceptable salt thereof, or a mixture of these.

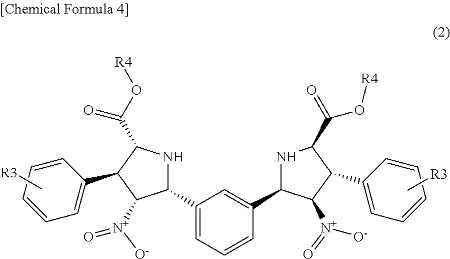

In Formula (2), R3 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R4 each independently represent hydrogen, C1-C6 alkyl, C1-C6 halogenoalkyl, C2-C5 alkenyl, C3-C6 cycloalkyl, or optionally substituted aryl. One or more R3 may be present in the same ring.

A preferred substituent in the compound indicated by Formula (2) is hydrogen for each R3, and is methyl for each R4. A preferred compound of Formula (2) counting conformation is dimethyl 5,5′-(1,3-phenylene) (2R, 2′R, 3S, 3′S, 4R, 4′R, 5R, 5′R)-bis(4-nitro-3-phen ylpyrrolidine-2-carboxylate) (hereinafter, indicated by Compound 2 (No. 195)).

The compound indicated by Formula (2) can be produced, for example, for the case of Compound 2 (No. 195), by the step of reacting 2,6-bis((2R,4S,5S)-1-benzyl-4,5-diphenylimidazolidine-2-yl)pyridine and copper (II) trifluoromethanesulfonate in an appropriate solvent such as methylene chloride, and the step of adding to this reaction solution, (E)-(2-nitrovinyl)benzene, 1,4-dioxane, triethylamine, and dimethyl 2,2′-(((1E′E)-1,3-phenylenebis′(methanylylidene))bis(azanylylidene)) diacetate and letting them react.

As the solvent used in the above production method, solvents that are similar to those described for the compound indicated by Formula (1) can be selected. In the above production method, each compound may be protected with an appropriate protective agent that is known by a person skilled in the art as is necessary. Such a protective agent and deprotecting reaction can be appropriately selected and used by a person skilled in the art according to the substituent to be protected.

### Antiviral Agents

The present invention provides a drug containing as an active ingredient a compound selected from the group consisting of the compounds indicated by Formulas (1) to (2), and Compounds 3 to 22 listed in the following Table 1 to Table 6, an isomer thereof, a pharmaceutically acceptable salt thereof, or a mixture of these.

TABLE 1

| | Compound No. in | |
|---|---|---|
| Compound | Chiba University | |
| No. | Compound Library | Name of compound |
| 3 | No. 1 | 2-(5-(5-(5-ferrocenylthiophene-2-yl)-1-phenyl- |
| | | 1H-pyrrol-2-yl)thiophene-2-yl)ethene-1,1,2- |
| | | tricarbonitrile |
| | | 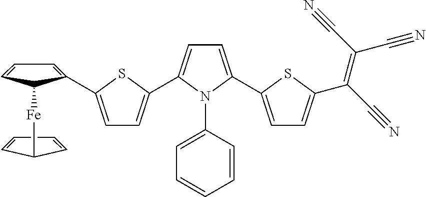 |
| 4 | No. 2 | (R)-2-amino-2-(naphthalene-1-yl) acetic acid |
| | | 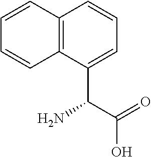 [SMILES: N[C@@H](C(=O)O)c1cccc2ccccc12] |
| 5 | No. 6 | N1,N2-diphenylethane-1,2-diamine |
| | | 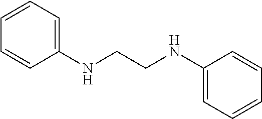 [SMILES: c1ccc(NCCNc2ccccc2)cc1] |
| 6 | No. 7 | N1,N2-bis(4-methoxyphenyl)ethane-1,2-diamine |
| | | 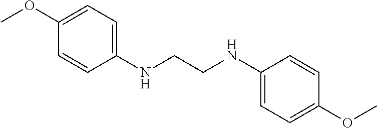 |
| 7 | No. 8 | N1,N2-bis(4-chlorophenyl)ethane-1,2-diamine |
| | | 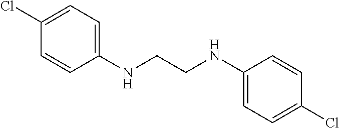 [SMILES: Clc1ccc(NCCNc2ccc(Cl)cc2)cc1] |

TABLE 2

| | Compound No. in | |
|---|---|---|
| Compound | Chiba University | |
| No. | Compound Library | Name of compound |
| 8 | No. 9 | (4S,11R)-1-phenyl-4-((phenylamino) |
| | | methyl)-1,2,3,4,6-pentahydrobenzo |
| | | [3,4][1,2]azaphosphoro[1,2-a][1,3,2] |
| | | diazaphosphosphinine 11-oxide |
| | | 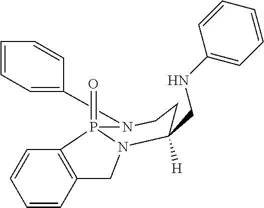 |
| 9 | No. 62 | (S)-3-(1H-indole-3-yl)-1-methyl-3- |
| | | (nitromethyl)indoline-2-one |
| | | 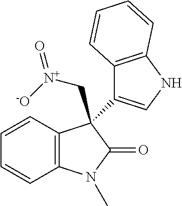 |
| 10 | No. 70 | (S)-1,5-dimethyl- |
| | | 3-(7-methyl-1H-indole-3-yl)-3-(nitromethyl) |
| | | indoline-2-one |
| | | 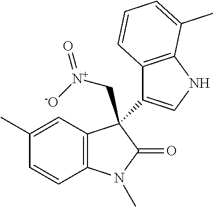 |

TABLE 3

| | Compound No. in | |
|---|---|---|
| Compound | Chiba University | |
| No. | Compound Library | Name of compound |
| 11 | No. 80 | 2,6-bis((2R,4S,5S)-1-benzyl-4,5- |
| | | diphenylimidazolidine-2-yl)pyridine |
| | | 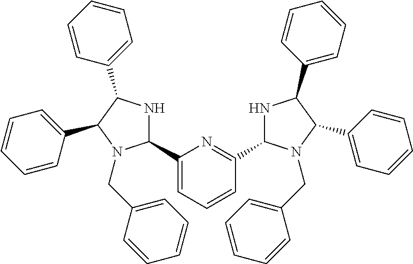 [SMILES: c1ccc(CN2[C@H](c3cccc([C@@H]4N[C@@H](c5ccccc5)[C@H](c5ccccc5)N4Cc4ccccc4)n3)N[C@@H](c3ccccc3)[C@@H]2c2ccccc2)cc1] |
| 12 | No. 124 | tert-butyl(2′R,3R,4′S,5′S)-4′-(4-bromophenyl)- |
| | | 2-oxo-2′-phenylspiro[indoline-3,3′-pyrrolidine]- |
| | | 5′-carboxylate |
| | | 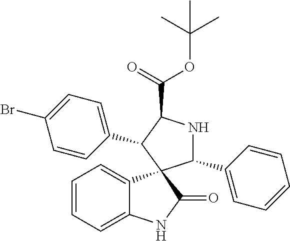 [SMILES: CC(C)(C)OC(=O)[C@H]1N[C@H](c2ccccc2)[C@@]2(C(=O)Nc3ccccc32)[C@@H]1c1ccc(Br)cc1] |
| 13 | No. 125 | tert-butyl(2′R,3R,4′S,5′S)-2-oxo-2′-phenyl-4′- |
| | | (p-tolyl)spiro[indoline-3,3′-pyrrolidine]-5′- |
| | | carboxylate |
| | | 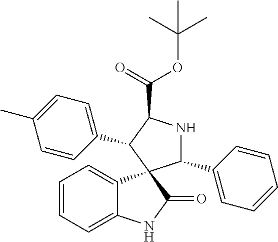 [SMILES: Cc1ccc([C@@H]2[C@@H](C(=O)OC(C)(C)C)N[C@H](c3ccccc3)[C@@]23C(=O)Nc2ccccc23)cc1] |

TABLE 4

| | Compound No. in | |
|---|---|---|
| Compound | Chiba University | |
| No. | Compound Library | Name of compound |
| 14 | No. 199 | (E)-1-allyl-3-(nitromethylene) |
| | | indoline-2-one |
| | | 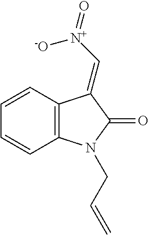 |
| 15 | No. 221 | 2,4-dibromo-6-((E)-(((1R,2R)-2- |
| | | (isoindoline-2-yl)-1,2-diphenylethyl) |
| | | imino)methyl)phenol |
| | | 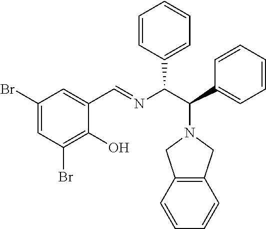 [SMILES: Oc1c(Br)cc(Br)cc1/C=N/[C@H](c1ccccc1)[C@@H](c1ccccc1)N1Cc2ccccc2C1] |
| 16 | No. 225 | 2-(phenyl((4-(trifluoromethoxy)phenyl) |
| | | amino)methyl)cycloheptane-1-one |
| | | 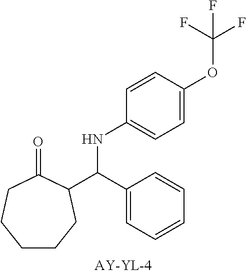 [SMILES: O=C1CCCCCC1C(Nc1ccc(OC(F)(F)F)cc1)c1ccccc1] |

TABLE 5

| | Compound No. in | |
|---|---|---|
| Compound | Chiba University | |
| No. | Compound Library | Name of compound |
| 17 | No. 239 | ethyl(R)-1-(3-chlorophenyl)-5-oxo- |
| | | 6,7-dihydro-1H,5H-pyrazolo |
| | | [1,2-a]pyrazole-2-carboxylate |
| | | 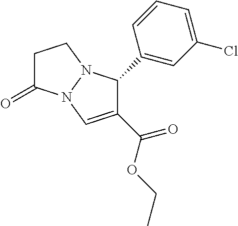 [SMILES: CCOC(=O)C1=CN2C(=O)CCN2[C@@H]1c1cccc(Cl)c1] |
| 18 | No. 240 | ethyl(R)-1-(4-methoxyphenyl)-5-oxo- |
| | | 6,7-dihydro-1H,5H-pyrazolo |
| | | [1,2-a]pyrazole-2-carboxylate |
| | | 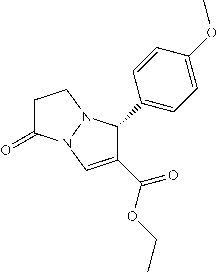 [SMILES: CCOC(=O)C1=CN2C(=O)CCN2[C@@H]1c1ccc(OC)cc1] |
| 19 | No. 242 | ethyl(R)-1-cyclohexyl-5-oxo-6,7- |
| | | dihydro-1H,5H-pyrazolo |
| | | [1,2-a]pyrazole-2-carboxylate |
| | | 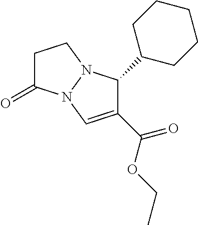 [SMILES: CCOC(=O)C1=CN2C(=O)CCN2[C@@H]1C1CCCCC1] |

TABLE 6

| | Compound No. in | |
|---|---|---|
| Compound | Chiba University | |
| No. | Compound Library | Name of compound |
| 20 | No. 279 | methyl(2S,3R,4S,5S)-4-nitro-3,5- |
| | | diphenylpyrrolidine-2-carboxylate |
| | | 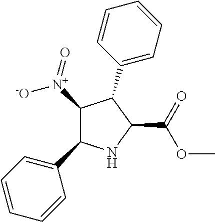 |
| 21 | No. 302 | 2,5-diphenyl-1,3,4-oxadiazole |
| | | 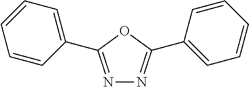 [SMILES: c1ccc(-c2nnc(-c3ccccc3)o2)cc1] |
| 22 | No. 306 | ethyl |
| | | 7,7-dimethyl-5-oxo-l-phenyl- |
| | | 6,7-dihydro-1H,5H-pyrazolo |
| | | [1,2-a]pyrazole-2-carboxylate |
| | | 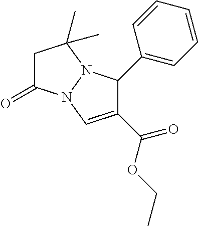 [SMILES: CCOC(=O)C1=CN2C(=O)CC(C)(C)N2C1c1ccccc1] |

Compound 3 (No. 1) is the compound 3 that was disclosed in Abstracts of presentations of The Chemical Society of Japan, The 85 the Spring Annual Meeting (issued on Mar. 11, 2005, p. 1355, presentation number 4F7-27), and in the presentation, and can be produced by coupling between 1-aryl-2,5-di(2-thienyl)pyrrole (wherein aryl is phenyl, one of the thienyls has tricyanoethenyl) and ferrocenylboronic acid.

Compound 4 (No. 2) can be produced according to the method described in Bulletin of the Chemical Society of Japan, 1992, Vol. 65 (No. 9), pp. 2359-2365.

Compound 5 (No. 6) is the compound 18 described in J. Org. Chem., 2006, Vo. 71, pp. 4481-4489, and can be produced according to the method described in the same literature.

Compound 6 (No. 7) is an intermediate of scheme 5 described in page 7990 of Tetrahedron, 2010, Vol. 66, pp. 7988-7994, and can be produced according to the method described in the same literature.

Compound 7 (No. 8) is an intermediate of scheme 6 (X is Cl) described in page 7990 of Tetrahedron, 2010, Vol. 66, pp. 7988-7994, and can be produced according to the method described in the same literature.

Compound 8 (No. 9) is the compound 3 described in Tetrahedron: Asymmetry, 2007, Vol. 18, pp. 1844-1849, and can be produced according to the method described in the same literature.

Compound 9 (No. 62) is the compound (2-1) described in Example 1 of JP 2013-142080 A, and can be produced according to the method described in the same publication.

Compound 10 (No. 70) corresponds to a compound described in Formula (2) in JP 2013-142080 A, in which each of X, Y and R1 is methyl, and R2 is a hydrogen atom. This compound can also be produced according to the method described in the same publication.

Compound 11 (No. 80) is a compound registered as CAS No. 1223020-29-8, and is commercially available, for example, as a product code B3934 from Tokyo Chemical Industry Co., Ltd.

Compound 12 (No. 124) and Compound 13 (No. 125) are the compound 3c and compound 3e described in Table 2 of Chem. Euro. J., 2012, Vol. 18, pp. 8278-8282, respectively, and both of these can be produced according to the method described in the same literature.

Compound 14 (No. 199) can be produced according to the method described in A. Angew. Chem. Int. Ed., 2013, Vol. 52, pp. 2486-2490 or Chemical Paper, 2010, Vol. 64, pp. 673-677.

Compound 15 (No. 221) can be produced according to the method described in Eur. J. Inorg. Chem., 2013, pp. 2093-2101.

Compound 16 (No. 225) can be produced according to the method described in Org. Lett., 2014, Vol. 16, pp. 86-89.

Each of Compound 17 (No. 239), Compound 18 (No. 240) and Compound 19 (No. 242) can be produced according to the method described in Chem. Commun., 2013, Vol. 49, pp. 7776-7778.

Compound 20 (No. 279) can be produced according to the method described in J. Am. Chem. Soc., 2010, Vol. 132, pp. 5338-5339.

Compound 21 (No. 302) is a compound registered as CAS No. 725-12-2, and is commercially available, for example, as a product code D1429 from Tokyo Chemical Industry Co., Ltd.

Compound 22 (No. 306) can be produced according to the method described in Chem. Commun., 2013, Vol. 49, pp. 7776-7778.

As shown in the later-described Examples, it was confirmed that each of the compounds indicated by the above Formulas (1) and (2) and Compounds 3 to 22 showed antiviral activity in the MTT assay. The present invention provides an antiviral agent containing as an active ingredient, a compound indicated by the Formula (1) or (2), or Compounds 3 to 22, an isomer thereof, a pharmaceutically acceptable salt thereof or a mixture of these.

Examples of the pharmaceutically acceptable salt include salts in ordinarily known basic groups such as an amino group, or acidic groups such as a hydroxyl group, a phosphoryl group or a carboxyl group.

Examples of the salts in basic groups include salts with mineral acid such as hydrochloric acid, hydrobromic acid and sulfuric acid, salts with organic carboxylic acid such as tartaric acid, formic acid, citric acid, trichloroacetic acid and trifluoroacetic acid, and salts with sulfonic acid such as methanesulfonic acid, benzenesulfonic acid, p-toluenesulfonic acid, mesitylenesulfonic acid and naphthalenesulfonic acid.

Examples of the salts in acidic groups include salts with alkali metal such as sodium and potassium; salts with alkali earth metal such as calcium and magnesium; ammonium salts; and salts with a nitrogen-containing organic base such as trimethylamine, triethylamine, tributylamine, pyridine, N,N-dimethylaniline, N-methylpiperidine, N-methylmorpholine, diethylamine, dicyclohexylamine, procaine, dibenzylamine, N-benzyl-β-phenethylamine, 1-ephenamine and N,N′-dibenzylethylene diamine.

Among the above salts, pharmaceutically acceptable salts are preferred.

Preferred examples of the antiviral agent in the present invention include antiviral agents against Lassa virus, influenza virus, norovirus, SARS virus, rubella virus, mumps virus, measles virus, RS virus, polio virus, ECHO virus, Coxsackie virus, Ebola virus, dengue virus, Newcastle disease virus, hepatitis C virus, rabies virus, or human immunodeficiency virus. More preferred examples include antiviral agents against RS virus or influenza virus, and the most preferred example is an antiviral agent against RS virus.

The compound or the antiviral agent in the present invention can be prepared into a pharmaceutical composition by mixing various pharmaceutical additives such as an excipient, a binder, a disintegrator, a collapse suppressor, an anticaking/antibonding agent, a lubricant, an absorption/adsorption carrier, a solvent, an extender, an isotonizing agent, a solubilizing agent, an emulsifier, a suspending agent, a thickener, a coating, an absorption promoter, a gelation/solidification promotor, a photo-stabilizer, a preservative, a desiccating agent, an emulsification/suspension/dispersion stabilizer, an anti-coloring agent, a deoxidant/antioxidant, a corrigent, a coloring agent, a foaming agent, a defoaming agent, a soothing agent, an antistatic agent, and a buffer/pH regulator, and further, pharmaceutical preparations such as oral preparations (tablets, capsules, powders, granules, fine granules, pills, suspensions, emulsions, liquids and solutions, syrups, etc.), injections, suppositories, external preparations (ointments, patches, etc.), and aerosols containing these can be prepared.

Oral solid preparations such as tablets, powders, and granules can be prepared according to ordinary methods by using pharmaceutical additives for solid preparation including, for example, excipients such as lactose, white soft sugar, sodium chloride, glucose, starch, calcium carbonate, kaolin, crystalline cellulose, anhydrous dicalcium phosphate, partially pregelatinized starch, cornstarch and alginic acid; binders such as simple syrup, glucose liquid, starch liquid, gelatin solution, polyvinyl alcohol, polyvinyl ether, polyvinyl pyrrolidone, carboxymethyl cellulose, shellac, methyl cellulose, ethyl cellulose, sodium alginate, gum arabic, hydroxypropylmethyl cellulose, hydroxypropyl cellulose, water and ethanol; disintegrators such as dry starch, alginic acid, agar powder, starch, cross-linked polyvinyl pyrrolidone, cross-linked sodium carboxymethylcellulose, calcium carboxymethylcellulose and sodium carboxymethyl starch; collapse suppressors such as stearyl alcohol, stearic acid, cocoa butter and hydrogenated oil; anticaking/antibonding agents such as aluminum silicate, calcium hydrogenphosphate, magnesium oxide, talc, and silicic anhydride; lubricants such as carnauba wax, light anhydrous silicic acid, aluminum silicate, magnesium silicate, hardened oil, hardened vegetable oil derivatives, sesame oil, white beeswax, titanium oxide, dried aluminum hydroxide gel, stearic acid, calcium stearate, magnesium stearate, talc, calcium hydrogenphosphate, sodium lauryl sulfate and polyethylene glycol; absorption promoters such as quaternary ammonium salts, sodium lauryl sulfate, urea and enzymes; and absorption/adsorption carriers such as starch, lactose, kaolin, bentonite, silicic anhydride, hydrated silicon dioxide, magnesium aluminometa silicate and colloidal silicic acid.

Further, tablets can be prepared as normal tablets with coating, for example, sugar-coated tablets, gelatin-coated tablets, gastrosoluble coated tablets, enteric coated tablets and water-soluble film coated tablets as necessary.

Capsules can be prepared by encapsulating the various pharmaceutical compositions illustrated above in a hard gelatin capsule or a soft capsule.

Alternatively, aqueous or oily suspensions, solutions, syrups and elixirs can be prepared by preparation according to ordinary methods using the afore-mentioned various additives for liquid preparations such as a solvent, an extender, an isotonizing agent, a solubilizing agent, an emulsifier, a suspending agent, and a thickener.

Suppositories can be prepared by adding an appropriate absorption promoter to, for example, polyethylene glycol, cacao butter, lanolin, higher alcohols, esters of higher alcohol, gelatin, semisynthetic glyceride or Witepsol.

Injections can be prepared according to ordinary methods by using pharmaceutical additives for liquid preparation, including, for example, diluents such as water, ethyl alcohol, macrogol, propylene glycol, citric acid, acetic acid, phosphoric acid, lactic acid, sodium lactate, sulfuric acid and sodium hydroxide; pH regulators and buffers such as sodium citrate, sodium acetate and sodium phosphate; stabilizers such as sodium pyrosulfite, ethylenediaminetetraacetic acid, thioglycolic acid and thiolactic acid; isotonizing agents such as common salt, glucose, mannitol or glycerin; solubilizing agents such as carboxymethyl cellulose sodium, propylene glycol, sodium benzoate, benzyl benzoate, urethane, ethanolamine, and glycerin; soothing agents such as calcium gluconate, chlorobutanol, glucose, and benzyl alcohol; and topical anesthetics.

Ointments in the forms of paste, cream and gel can be formulated by mixing according to ordinary methods by using pharmaceutical additives including, for example, bases such as white petrolatum, polyethylene, paraffin, glycerin, cellulose derivatives, polyethylene glycol, silicon and bentonite; preservatives such as methyl, parahydroxybenzoate, ethyl parahydroxybenzoate, and propyl parahydroxybenzoate; stabilizers; and moistening agents.

In producing patches, the above ointment, cream, gel or paste can be applied on ordinary supports by ordinary methods. Examples of the supports include woven fabrics or nonwoven fabrics formed of cotton, staple fiber and chemical fiber; and films or foam sheets of soft polyvinyl chloride, polyethylene and polyurethane can be used.

The administration method of the pharmaceutical preparations is not particularly limited, and is appropriately determined depending on the form of the preparation, age, sex or other conditions of the patient, and the degree of symptoms of the patient.

The dose of the active ingredient of the drug of the present invention is appropriately selected depending on the direction for use of the drug, age and sex of the patient, the form of the disease, and other conditions, and for an adult, generally 0.1 to 100 mg/kg per day can be administered in a single dose or multiple dose.

Hereinafter, the present invention will be described more specifically by way of Examples. In Examples, the mixing ratio in an eluate is indicated by a volume ratio. As the carrier in the column chromatography, silica gel BW-127ZH (available from FUJI SILYSIA CHEMICAL LTD.) was used; as the carrier in the reversed phase silica gel column chromatography, YMC/GEL ODS-AM 120-S50 (YMC CO., LTD.) was used; and as the carrier in the ion exchange column chromatography is DEAE cellulose (Wako Pure Chemical Industries, Ltd.) was used.

### EXAMPLES

### Example 1 Synthesis of Compound 1 (No. 150)

### Synthesis of dimethyl 5-(dicyanomethyl)-2-hydroxy-4,5-diphenylcyclopenta-1,3-diene-1,3-dic arboxylate

In a solution containing dimethyl 2-oxo-4,5-diphenylcyclopenta-3,5-diene-1,3-dicarboxylate (0.348 g) and malononitrile (0.069 g) in chloroform (2 mL), aluminum oxide (0.204 g) was added in several batches, and the mixture was allowed to react at room temperature for 2 hours. The reaction mixture was filtered through celite using chloroform, and the filtrate was concentrated. The residue was re-crystallized in a chloroform-hexane mixed solution to obtain the title compound (0.308 g) having the following physical properties. TLC: Rf 0.2 (hexane:ethyl acetate=2:1); NMR (CDCl₃): δ 3.70 (s, 3H), 3.74 (s, 3H), 5.04 (s, 1H), 6.84 (d, J=7.3 Hz, 2H), 7.21-7.29 (m, 4H), 7.39-7.43 (m, 4H).

### Example 2 Synthesis of Compound 2 (No. 195)

### Synthesis of dimethyl 5,5′-(1,3-phenylene) (2R, 2′R, 3S, 3′S, 4R, 4′R, 5R, 5′R)-bis(4-nitro-3-phen ylpyrrolidine-2-carboxylate)

In a methylene chloride (0.75 mL) solvent, 2,6-bis((2R, 4S, 5S)-1-benzyl-4,5-diphenylimidazolidine-2-yl)pyridine (22.17 mg) and copper (II) trifluoromethanesulfonate (10.85 mg) were stirred at room temperature for 2 hours, and the mixture solution was concentrated. To the reaction mixture, (E)-(2-nitrovinyl)benzene (44.75 mg), 1,4-dioxane (0.75 mL), triethylamine (4.19 μL), and dimethyl 2,2′-(((1E, 1′E)-1,3-phenylenebis(methanylylidene))bis(azanylylidene)) diacetate (41.44 mg) were added, and the mixture was allowed to react at room temperature for 24 hours. Water (5 mL) was added to the reaction mixture, and the mixture was concentrated. The residue was extracted three times with ethyl acetate. The organic layer was washed with saturated brine, dried over anhydrous magnesium sulfate, and then concentrated. The residue was purified by silica gel chromatography (hexane:ethyl acetate=2:1), to obtain the title compound (60.3 mg) having the following physical properties.

TLC: Rf 0.15 (hexane:ethyl acetate=2:1); NMR (CDCl₃): δ 3.31 (m, 2H), 3.81 (s, 6H), 4.15 (t, 2H), 4.22 (dd, J=3.85, 7.48 Hz, 2H), 4.92 (dd, J=6.57, 9.97 Hz, 2H), 5.30 (m, 2H), 7.36 (m, 14H).

### Example 3 Synthesis of Compound 3 (No. 1)

### Synthesis of 2-(5-(5-(5-ferrocenylthiophene-2-yl)-1-phenyl-1H-pyrrol-2-yl)thiophe ne-2-yl)ethene-1,1,2-tricarbonitrile

In a solution of 1-phenyl-2,5-di(thiophene-2-yl)-1H-pyrrole (0.707 g) in tetrahydrofuran (7 mL), n-butyllithium (1.6 M hexane solution, 1.9 mL) was added at −78° C. and allowed to react for 30 minutes. Iodine (0.642 g) was added to the reaction solution, and allowed to react for 30 minutes. To the reaction mixture, saturated sodium thiosulfate (5 mL) was added, and extracted three times with chloroform. The organic layer was washed with saturated brine, dried over anhydrous magnesium sulfate, and concentrated, and the residue was purified with silica gel chromatography (hexane), to obtain 2-(5-iodethiophene-2-yl)-1-phenyl-5-(thiophene-2-yl)-1H-pyrrole (0.638 g) having the following physical properties.

NMR (CDCl₃): δ 6.19 (d, J=3.8 Hz, 1H), 6.50 (d, J=3.8 Hz, 1H), 6.51 (dd, J=1.1 and 3.6 Hz, 1H), 6.52 (d, J=4.1 Hz, 1H), 6.80 (dd, J=3.7 and 5.3 Hz, 1H), 6.93 (d, J=3.8 Hz, 1H), 7.05 (dd, J=1.2 and 5.3 Hz, 1H), 7.29 (dd, J=1.6 and 8.4 Hz, 2H), 7.40-7.51 (m, 3H).

In a solution of 2-(5-iodethiophene-2-yl)-1-phenyl-5-(thiophene-2-yl)-1H-pyrrole (0.650 g) in a mixed solution (3 mL) of dimethoxyethane and 3 M NaOH aqueous solution, tetrakis(triphenylphosphine)palladium (0.273 g) was added, and allowed to react under reflux in an argon atmosphere for one day. Water (5 mL) was added to the reaction mixture, and extracted three times with chloroform. The organic layer was washed with saturated brine, dried over anhydrous magnesium sulfate, and then concentrated. The residue was dissolved in tetrahydrofuran (5 mL), and n-butyllithium (1.6 M hexane solution, 0.6 mL) was added at −78° C. and allowed to react for 30 minutes. Tetracyanoethylene (0.218 g) was added to the reaction solution and allowed to react for 15 minutes. To the reaction mixture, saturated ammonium chloride aqueous solution (5 mL) was added, and the mixture was extracted three times with chloroform. The organic layer was washed with saturated brine, dried over anhydrous magnesium sulfate, and concentrated, and the residue was purified by silica gel chromatography (chloroform) to obtain the title compound (0.107 g) having the following physical properties.

NMR (CDCl₃): δ 4.03 (s, 5H), 4.28 (t, J=1.9 Hz, 2H), 4.46 (t, J=1.9 Hz, 2H), 6.53 (d, J=4.0 Hz, 1H), 6.74-6.75 (m, 2H), 6.97 (d, J=4.5 Hz, 1H), 7.10 (d, J=4.3 Hz, 1H), 7.48 (d-like, J=6.9 Hz, 2H), 7.61-7.73 (m, 3H), 7.75 (d, J=4.5 Hz, 1H).

### Example 4 Synthesis of Compound 8 (No. 9)

### Synthesis of (4S,11R)-1-phenyl-4-((phenylamino)methyl)-1,2,3,4,6-pentahydrobenzo[3,4][1,2]azaphosphoro [1,2-a][1,3,2]diazaphosphinine 11-oxide

A solution of 8-(2-iodebenzyl)-2,7-diphenyl-2,7,8-triaza-1-phosphabicyclo[3.2.1]oc tane 1-oxide (103 mg) in THF (3 mL) was cooled to −78° C., and then a solution of 1.5 M n-butyllithium in hexane (0.146 mL) was added, and the mixture was stirred at a temperature of −60° C. or lower for 1.5 hours. After adding sodium sulfate decahydrate to the reaction liquid and stirring for 10 minutes, the reaction liquid was diluted with ethyl acetate. The obtained solution was washed with water and saturated brine, dried over anhydrous sodium sulfate, and then concentrated. The residue was purified by silica gel chromatography (hexane:ethyl acetate=2:1 →1:2), to obtain the title compound (60.6 mg) having the following physical properties.

¹H NMR (CDCl₃): δ 2.28-2.36 (m, 1H), 2.49-2.52 (m, 1H), 3.41-3.60 (m, 4H), 3.73-3.80 (m, 1H), 4.20 (dd, J=6.4 (PNCH), 14.0 Hz, 1H), 4.63 (broad peak (NH), 1H), 4.71 (dd, J=6.4 (PNCH), 14.0 Hz, 1H), 6.69-6.72 (m, 4H), 7.04-7.07 (m, 1H), 7.17-7.21 (m, 2H), 7.25-7.46 (m, 6H), 7.56-7.60 (m, 1H).

¹³C NMR (CDCl₃): δ 30.3 (d, J=6.9 Hz), 47.2, 48.3, 55.3 (d, J=17.2 Hz), 56.7, 113.0 (2C), 117.3, 120.4 (d, J=4.2 Hz)(2C), 122.8, 123.6 (d, J=10.7 Hz), 127.6 (d, J=11.9 Hz), 127.9 (d, J=13.0 Hz), 129.2 (2C), 129.3 (2C), 131.4 (d, J=2.6 Hz), 141.3 (d, J=22.1 Hz), 145.0, 148.1.

³¹P NMR (CDCl3): δ 26.0.

### Example 5 Synthesis of Compound 15 (No. 221)

### Synthesis of 2,4-dibromo-6-((E)-(((1R,2R)-2-(isoindoline-2-yl)-1,2-diphenylethyl) imino)methyl)phenol

To a solution of (1R,2R)-2-(isoindoline-2-yl)-1,2-diphenylethane-1-amine (518.8 mg) in dichloromethane (10 mL), anhydrous sodium sulfate (9.0 mg) and 3,5-dibromosalicylaldehyde (419.9 mg) were added, and the mixture was allowed to react at room temperature for 12 hours. The reaction mixture was filtered, and concentrated. The residue was purified by silica gel chromatography (hexane:ethyl acetate=4/1), to obtain the title compound (860.6 mg) having the following physical properties.

TLC: Rf 0.57 (hexane:ethyl acetate=4:1); NMR (CDCl₃): δ 3.98 (d, J=11.00 Hz, 2H), 4.07 (d, J=11.00 Hz, 2H), 4.30 (d, J=7.02 Hz, 1H), 4.97 (d, J=7.02 Hz, 1H), 7.09-7.19 (m, 14H), 7.29 (s, 1H), 7.69 (s, 1H), 8.31 (s, 1H).

### Example 6 Synthesis of Compound 16 (No. 225)

### Synthesis of 2-(phenyl((4-(trifluoromethoxy)phenyl)amino)methyl)cycloheptane-1-on e

A mixture of silver trifluoromethanesulfonate (25.6 mg) and (R)-SEGPHOS (30.5 mg) was dissolved in tetrahydrofuran (1 mL) in the presence of molecular sieves 4 A (200 mg), and stirred at room temperature for 20 minutes. To this solution, 2,2,2-trifluoroethanol (108 μL) and N,N-diisopropylethylamine (34 μL) were added at −30° C., and stirred for 5 minutes. Then to this solution, a solution of cyclohept-1-ene-1-yl 2,2,2-trichloroacetate (257 mg) and 1-phenyl-N-(4-(trifluoromethoxy)phenyl)methaneimine (133 mg) in tetrahydrofuran (2 mL) was dropped at −30° C., and the mixture was allowed to react at this temperature for 16 hours. After adding methanol (2 mL) to the reaction mixture, and filtration through celite, the reaction mixture was concentrated. The residue was purified by silica gel chromatography (hexane:ethyl acetate=9:1), to obtain the title compound (99.4 mg) having the following physical properties. TLC: Rf 0.20 (hexane:ethyl acetate=6:1); NMR (DMSO-d6): δ 1.10-1.28 (m, 2H), 1.33-1.43 (m, 1H), 1.51-1.60 (m, 1H), 1.76-1.88 (m, 4H), 2.12-2.19 (m, 1H), 2.44-2.49 (m, 1H), 2.82-2.87 (m, 1H), 4.69 (t, J=7.2 Hz, 1H), 6.29 (d, J=8.9 Hz, 1H), 6.56 (d, J=9.2 Hz, 2H), 6.93 (d, J=8.3 Hz, 2H), 7.13 (t, J=7.2 Hz, 1H), 7.23 (t, J=8.0 Hz, 2H), 7.28 (d, J=8.0 Hz, 2H).

### Example 7 Evaluation of Anti-RS Viral Activity

The aforementioned Compound 1 (No. 150) to Compound 22 (No. 306) were measured for anti-RS viral activity according to the protocol of MTT assay kit (CellTiter-Glo (registered trade name) Assay, Promega Corporation).

As Compound 1 (No. 150), Compound 2 (No. 195), Compound 3 (No. 1), Compound 8 (No. 9), Compound 15 (No. 221) and Compound 16 (No. 225), those synthesized by the methods described in Examples 1 to 6 were used, as Compound 4 (No. 2), Compound 5 (No. 6), Compound 6 (No. 7), Compound 7 (No. 8), Compound 9 (No. 62), Compound 10 (No. 70), Compound 12 (No. 124), Compound 13 (No. 125), Compound 14 (No. 199), Compound 17 (No. 239), Compound 18 (No. 240), Compound 19 (No. 242), Compound 20 (No. 279) and Compound 22 (No. 306), those synthesized according to the description of the aforementioned literatures in which respective compounds are disclosed were used, and as Compound 11 (No. 80) and Compound 21 (No. 302), products commercially available from Tokyo Chemical Industry Co., Ltd. were used.

In a 96-well plate, Hep2 cells were seeded at 1×10³ cells per well, and each of compounds in the amount shown in Table 7, and RS virus having an infectivity titer of 1 MOI were added, and cultured for 72 hours, and then a viable cell count was determined. The results are shown in Table 7. As a control, a well containing DMSO at 25 μM (when the additive amount of the compound is 80 μM) or 5 μM (when the additive amount of the compound is 16 μM) in place of a compound was prepared.

TABLE 7

| Compound No. | Additive | Survival |
|---|---|---|
| (Compound No. in Chiba | Amount | Rate |
| University Compound Library) | (μM) | (%) |
| Control | 0 | 100 |

| | | | |
|---|---|---|---|
| 1 | (No. 150) | 80 | 359 |
| 2 | (No. 195) | 80 | 332 |
| 3 | (No. 1) | 16 | 541 |
| 4 | (No. 2) | 80 | 450 |
| 5 | (No. 6) | 16 | 471 |
| 6 | (No. 7) | 16 | 472 |
| 7 | (No. 8) | 16 | 405 |
| 8 | (No. 9) | 16 | 815 |
| 9 | (No. 62) | 80 | 276 |
| 10 | (No. 70) | 16 | 294 |
| 11 | (No. 80) | 16 | 274 |
| 12 | (No. 124) | 16 | 475 |
| 13 | (No. 125) | 16 | 406 |
| 14 | (No. 199) | 16 | 409 |
| 15 | (No. 221) | 16 | 393 |
| 16 | (No. 225) | 16 | 294 |
| 17 | (No. 239) | 80 | 264 |
| 18 | (No. 240) | 80 | 349 |
| 19 | (No. 242) | 16 | 209 |
| 20 | (No. 279) | 80 | 344 |
| 21 | (No. 302) | 80 | 248 |
| 22 | (No. 306) | 80 | 339 |

### Test Example Cytotoxicity Test

In the same manner as in Example 7, in a 96-well plate, Hep2 cells were seeded at 1×10³ cells per well, and each of Compounds 1 to 22 in variable amounts was added without addition of an RS virus, and cultured for 72 hours, and then a viable cell count was determined. Each compound showed 50% cell proliferation inhibitory concentration (IC50) of greater than or equal to 45 μM and greater than or equal to 216 μM in compounds added in an amount of 16 μM and in compounds added in an amount of 80 μM in the anti-RS viral activity test, respectively.

### INDUSTRIAL APPLICABILITY

The compounds provided by the present invention have the utility as drugs for the prevention or treatment of infectious diseases by virus, especially RS virus, and in particular infectious diseases in the lower airways (e.g., bronchiolitis, pneumonia, etc.).

1. (canceled)

2. (canceled)

3. A method for the prevention and/or treatment of a disease, comprising administering a human or an animal with an effective amount of a compound selected from the group consisting of the following compounds:
a compound indicated by the following Formula (1): 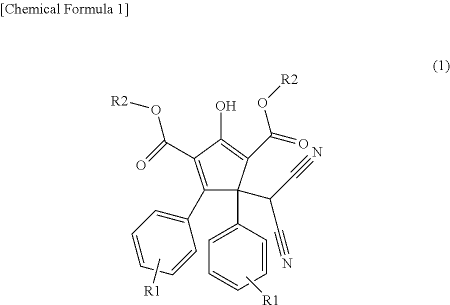
wherein R1 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R2 each independently represent hydrogen, C1-C6 alkyl, C1-C6 halogenoalkyl, C2-C5 alkenyl, C3-C6 cycloalkyl, optionally substituted aryl or heterocyclic group, and one or more R1 may be present in the same ring, an isomer thereof, a pharmaceutically acceptable salt thereof, or a mixture of these;
a compound indicated by the following Formula (2): 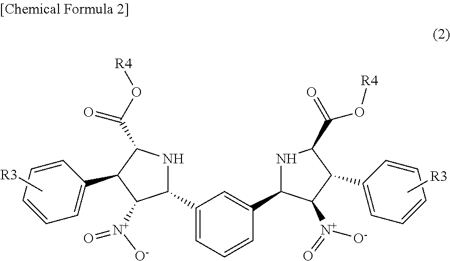
wherein R3 each independently represent hydrogen, halogen, hydroxyl, amino, carboxyl, C1-C6 alkyl, C1-C6 alkoxyl, C1-C6 halogenoalkyl, C1-C6 alkoxycarbonyl, C1-C6 alkylamino, C2-C5 alkenyl, C3-C6 cycloalkyl or optionally substituted aryl, R4 each independently represent hydrogen, C1-C6 alkyl, C1-C6 halogenoalkyl, C2-C5 alkenyl, C3-C6 cycloalkyl, or optionally substituted aryl, and one or more R3 may be present in the same ring, an isomer thereof, a pharmaceutically acceptable salt thereof, or a mixture of these;
Compound 3
2-(5-(5-(5-ferrocenylthiophene-2-yl)-1-phenyl-1H-pyrrol-2-yl)thiophene-2-yl)ethene-1,1,2-tricarbonitrile;
Compound 4
(R)-2-amino-2-(naphthalene-1-yl) acetic acid;
Compound 5
N1,N2-diphenylethane-1,2-diamine;
Compound 6
N1,N2-bis(4-methoxyphenyl)ethane-1,2-diamine;
Compound 7
N1,N2-bis(4-chlorophenyl)ethane-1,2-diamine;
Compound 8
(4S,11R)-1-phenyl-4-((phenylamino)methyl)-1,2,3,4,6-pentahydrobenzo[3,4][1,2]azaphosphoro [1,2-a][1,3,2]diazaphosphinine 11-oxide;
Compound 9
(S)-3-(1H-indole-3-yl)-1-methyl-3-(nitromethyl)indoline-2-one;
Compound 10
(S)-1,5-dimethyl-3-(7-methyl-1H-indole-3-yl)-3-(nitromethyl)indoline-2-one;
Compound 11
2,6-bis((2R,4S,5S)-1-benzyl-4,5-diphenylimidazolidine-2-yl)pyridine;
Compound 12
tert-butyl(2′R,3R,4′S,5′S)-4′-(4-bromophenyl)-2-oxo-2′-phenylspiro[indoline-3,3′-pyrrolidine]-5′-carboxylate;
Compound 13
tert-butyl(2′R,3R,4′S,5′S)-2-oxo-2′-phenyl-4′-(p-tolyl)spiro[indoline-3,3′-pyrrolidine]-5′-carboxylate;
Compound 14
(E)-1-allyl-3-(nitromethylene)indoline-2-one;
Compound 15
2,4-dibromo-6-((E)-(((1R,2R)-2-(isoindoline-2-yl)-1,2-diphenylethyl)imino)methyl)phenol;
Compound 16
2-(phenyl((4-(trifluoromethoxy)phenyl)amino)methyl)cycloheptane-1-one;
Compound 17
ethyl(R)-1-(3-chlorophenyl)-5-oxo-6,7-dihydro-1H,5H-pyrazolo[1,2-a]pyrazole-2-carboxylate;
Compound 18
ethyl(R)-1-(4-methoxyphenyl)-5-oxo-6,7-dihydro-1H,5H-pyrazolo[1,2-a]pyrazole-2-carboxylate;
Compound 19
ethyl(R)-1-cyclohexyl-5-oxo-6,7-dihydro-1H,5H-pyrazolo[1,2-a]pyrazole-2-carboxylate;
Compound 20
methyl(2S,3R,4S,5S)-4-nitro-3,5-diphenylpyrrolidine-2-carboxylate;
Compound 21
2,5-diphenyl-1,3,4-oxadiazole; and
Compound 22
ethyl7,7-dimethyl-5-oxo-1-phenyl-6,7-dihydro-1H,5H-pyrazolo[1,2-a]pyrazole-2-carboxylate, an isomer thereof, a pharmaceutically acceptable salt thereof, or a mixture of these.

4. The method according to claim 3, wherein the disease is a viral infectious disease.

5. The method according to claim 3, wherein the disease is a RS viral infectious disease.
